(19) United States
(12) Patent Application Publication
(10) Pub. No.: US 2021/0386538 A1
(43) Pub. Date: Dec. 16, 2021

(54) Accommodating Intraocular Lens with Combination of Variable Aberrations for Extension of Depth of Field

(71) Applicant: AKKOLENS INTERNATIONAL B.V., Breda (NL)

(72) Inventor: Michiel Christiaan Rombach, Breda (NL)

(21) Appl. No.: 17/283,366

(22) PCT Filed: Oct. 8, 2019
(86) PCT No.: PCT/NL2019/050669

§ 371 Date: Apr. 7, 2021

(30) Foreign Application Priority Data
Oct. 8, 2018 (NL) 2021778

(51) Int. Cl. A61F 2/16

(52) U.S. Cl. CPC A61F 2/164; A61F 2240/002; A61F 2/1632

(57) ABSTRACT

The invention relates to an accommodating intraocular lens, having an optical axis (3), with the lens comprising at least two optical elements (1, 2), and haptics to allow mutual translation of said elements (1, 2) in a direction substantially perpendicular to the optical axis (3), in which at least two of the elements (1, 2) each comprising free-form optical surfaces. The invention also relates to a combination of such lens and an apparatus adapted for measuring the optical power of an eye.

### CROSS-REFERENCE TO RELATED APPLICATIONS

This application is the United States national phase of International Application No. PCT/NL2019/050669 filed Oct. 8, 2019, and claims priority to The Netherlands Patent Application No. 2021778 filed Oct. 8, 2018, the disclosures of which are hereby incorporated by reference in their entirety.

### BACKGROUND OF THE INVENTION

### Field of the Invention

An accommodating lens provides a combination of variable defocus and desired variable depth of field, or, alternatively, a lens provides a combination of variable defocus and variable correction of an undesired variable optical aberration. Free-form optical surfaces for the variation of optical powers can be provided pre-operatively, during manufacturing, or, alternatively, post-operatively by laser treatment of the implanted lens.

### Description of Related Art

This document, the present document, discloses a modification of an accommodating intraocular lens, henceforth also: ‘lens’, to be implanted in the human eye. Detailed descriptions of such lenses and other modifications thereof are disclosed in numerous documents, for example from NL2012133, NL201242, EP1871299, EP1932492 and related documents, for designs of such AlOLs, and, for clinical results, Alió, in Am J Ophthamol 2016 April, 164: 37-48, but not restricted hereto, all of which documents and references made therein are considered to be part of the present document. The present document discloses variable focus lenses, henceforth: ‘lens’. The lens has an optical axis and at least two optical elements with each of the elements comprising at least one free-form optical surface, each element with preferably at least one Alvarez, cubic, optical surface. Such lens provides varying optical power, varying ‘defocus’, of which the degree of defocus depends on the degree of mutual movement of the optical elements in a direction largely perpendicular to the optical axis. In addition the variable focus lens also comprises a wavefront encoding phase mask, ‘mask’. The mask gradually changes the steepness of free-form optical surface, changes the power of the cubic term, and thus provides extended depth of field, ‘EDOF’. A cubic phase mask according to a constant cubic term, or, alternatively, a modified cubic phase mask according to a gradually changing, for example, increasing cubic term, is the preferred embodiment for the mask. Variable EDOF can also be achieved by alternative mask designs according to optical surfaces of a combination of other surfaces of other Zernike orders.

For example, at least two free-form optical surfaces for variable aspheric aberration which provide variable aspherity, provide spherical aberration, can be added to the, for example, cubic surfaces which provide the variable defocus. For the effect of spherical aberration on depth of field refer to, for example, Mouroulis, “Depth of field extension with spherical optics”, Optics Express, August 2008, Vol. 16, No. 17.

Cubic wavefront encoding phase masks for EDOF are best known to extend depth of field, as set forth in, for example, technical applications, as in EP2110702 and in U.S. Pat. No. 6,547,139. The main invention of cubic phase encoding is reported in E. R. Dowski, Jr. and W. T. Cathey, “Extended depth of field through wavefront coding,” Appl. Opt. 34(11), 1859-1866 (1995) and in a number of accompanying patent documents, for example, in U.S. Pat. No. 5,748,371 and WO9957599. The mask preferably modifies phase of light and not amplitude of light. The mask can be a cubic mask modifying the cubic term, or, alternatively, an aspheric mask, modifying the apheric term, but not limited hereto as long as the optical mask keeps the optical transfer function extended. This extension can be fixed extension, or, alternatively, as set forth in the present document, a variable extension of which the range of extension depends on the shift of the mask largely perpendicular to the optical axis.

Note that EDOF extends focus but that EDOF, by definition, also increases blur and thus loss of contrast especially for higher spatial frequencies. EDOF widens the ‘focal tunnel’, the range of acceptable focus along the optical axis, which tunnel changes from ‘short to narrow’, meaning: sharp focus over a short DOF, to ‘longer and wider’, meaning: blurred focus over a longer DOF. Whether the advantage of the degree of EDOF outweighs the disadvantage of the loss of the contrast depends on the requirements of the specific application of the lens which degrees can be calculated and incorporated in the design of the mask. For example, the human vision requires sharp vision, a vision of high visual acuity of, say, >0.8, for distance vision while the acuity can drop to, say, 0.4 for close up reading. An accommodating intraocular lens providing such ‘asymmetric EDOF’ can be designed by methods as disclosed in the present document.

The movement of the optical elements for the purpose of, for example, accommodating intraocular lenses can be achieved by, but not restricted to, principles as disclosed in US2009062912 and WO2005084587, and the same concept, with various adaptations in, for example, US2014074233, WO2014058316, EP2765952, NL2012257278, US2010131955, US2010106245 and NL1029548. These principles have been shown to function well for camera applications as well for the human eye, for both spectacles as well as for accommodating intraocular lenses.

An application of such accommodating intraocular lenses is in ophthalmology, for example for spectacles and for intraocular lenses implanted in the human eye. Such accommodating, translating, intraocular lens constructions are known from other documents, for example from NL2012133, NL201242, EP1871299, EP1932492, for designs of such constructions, and, for clinical results, for example, Alio et al., in Am J Ophthamol 2016 April, 164: 37-48.

### SUMMARY OF THE INVENTION

This document discloses a lens which provides a combination of variable defocus and desired variable depth of field or, alternatively, which lens provides a combination of variable defocus and variable correction of an undesired variable optical aberration.

The lens has an optical axis and comprises at least two optical elements of which at least one element translates in at least one direction largely perpendicular to the optical axis. Each optical element comprises at least one free-form optical surface with the combination of surfaces provide variation of at least one optical aberration of the lens with a degree of variation which is dependent on the degree of shift of the at least one of the optical elements. Such translating lenses are known from said references with these lenses generally, but not necessarily so, comprising cubic optical surfaces to provide variable defocus power. Such cubic surfaces, additional optical surfaces and combinations of surfaces can be defined by Zernike polynomials, of which the first fifteen modes are considered to be relevant to the human eye, which polynomials are mentioned in the present document to explain the free-form shape of various optical surface shapes. Note that such optical shapes, such free-form, rotationally asymmetrical, optical surfaces can also be derived from, for example, spline, or Cartesian or NURBS algorithms or to any other algorithms. Free-form surfaces derived from any formula or algorithm are considered to be included in the present document. An example of such a cubic surface is a surface according to the formula

t₁=A(xy²+⅓³)+E.

The optics of such lens provides, firstly, a variable focus to provide accommodation of the eye, meaning: providing variable defocus to correct for defocuses aberrations of the eye to provide sharp vision from far to near, say, to reading distance. The translation of at least one of the optical elements is a shift, meaning: sliding, of the element in a direction perpendicular to the optical axis, or, alternatively, a rotation in a plane perpendicular to the optical axis, or, alternatively, a wedging of the optical elements, or, alternatively, any combination of any movements movement in a plane largely perpendicular to the optical axis.

The lens can comprise at least one anchoring haptic, meaning: a mechanical component to provide positioning and anchoring of the optical elements in the anterior chamber or, alternatively, the posterior chamber of the eye. The lens can comprise at least one translation haptic, meaning: a mechanical component to provide translation of at least one optical element by transfer of movement of at least one component in the eye to at least one of the optical elements. The lens can comprise at least one haptic to provide a combination of positioning and translation. The lens can comprise at least one haptic coupled to a natural component of the eye which component can be the ciliary mass of the eye, or, alternatively, the zonula network of the eye, or, alternatively, the capsular bag of the eye, or, alternatively, the iris of the eye, or, alternatively, any natural component of the eye. Also, the lens can be driven by liquid pressure generated in the posterior chamber of the eye, or, alternatively, the lens can be driven by MEMS, meaning: micro-electro-mechanical system, which MEMS to provide translation of at least one optical element. Furthermore, the lens can also comprise at least one optical surface to correct for any fixed optical disorder of the eye, for example, the fixed optical disorder presbyopia, also: reading far-sightedness.

Also, the lens can provide correction of at least one undesired variable optical disorder of the eye, for example variable astigmatism or, alternatively, variable spherical aberration, which undesired aberrations can be generated by any optical surface of the eye, or, alternatively, by any optical surface of the lens.

The lens can provide a combination of variable defocus increasing depth of field at near vision. Alternatively, the lens can provide a combination of variable defocus and variable spherical aberration, or, alternatively, variable astigmatism to correct for variable and undesired such aberrations of the eye. For example, the human eye is known to generate variable spherical aberration during natural accommodation. The cornea of the eye has been shown to provide a positive aspherical aberration which can change to a negative spherical aberration during accommodation. A lens designed according to lenses described in the present document could provide increasing positive aspherical aberration, Zernike modes Z₂², Z₂⁻², of which the degree of asphericity depends on the degree of mutual translation of the optical elements. So far vision is hardly affected and near vision, reading, supported. Variable correction by additional free-form optical surfaces of the lens can modify the total aspherical aberration of the eye in any desired direction and by the desired optical aspherical optical power. Such variable correction can be rotationally symmetrical, or, alternatively, can be of, a more complex, rotationally asymmetrical design. Variable comas, Zernike modes Z₃¹, Z₃⁻¹, can result, for example, from tilt of the lens versus the optical axis in the eye, with the main effect due to a tilt of the optical axis of the lens versus the optical axis of the cornea. Tilt of the accommodating intraocular lens can be due, for example, to a change in tilt of the lens during the accommodative process resulting in a change in the coma optical power. Such variable coma can be evaluated port operatively, for example, by OCT apparatus, by, for example measuring the degree of tilt, followed by calculation of the expected degree of coma and subsequently correct said variable coma by additional optical free-form surfaces which provide variable degree of correction of the coma enscribed by, for example, on the lens or in the lens, by post operative modification of the intraocular material by laser treatment. Variable astigmatisms, Zernike modes Z₂², Z₂⁻² can be evaluated port operatively, for example, by OCT apparatus, by, for example measuring the degree of tilt, followed by calculation of the expected degree of coma and subsequently correct said variable coma by additional optical free-form surfaces which provide variable degree of correction of the coma enscribed by, for example, on the lens or in the lens, by post operative modification of the intraocular material by laser treatment. Note that, of course, all variable aberrations can also be evaluated pre-operatively and corrections added to the lens during customized manufacturing.

The optical surfaces to provide the additional variable optical power can be added to the lens prior to implantation, during manufacturing of the lens, with the degree of variable optical power based on pre-operative measurements of the eye into which the lens is implanted. Alternatively, the optical surfaces to provide the additional variable optical power can be added to the lens after implantation, with the degree of variable optical power based on post-operative measurements of the eye into which the lens is implanted and with the modifications to the optical surfaces provided by, for example post operative modification of intraocular material by laser treatment as in, for example, WO2018152407, by a customized wavefront guided refractive laser treatment of the accommodating intraocular lens post-operatively. Such treatment can modify any, at least one, optical surface for variable aberration disclosed in the present document. Such treatments can also modify the optical surfaces providing variable defocus, for example, increase the steepness of these surfaces to increase the diopter change per unit of mutual translation of optical surfaces.

Preferably the additional free-form surfaces are adapted to provide a variable increase of additional optical power depending on the rate of mutual translation of the elements.

In an attractive embodiment the additional free-form surfaces are adapted to provide a variable decrease of additional optical power depending on the rate of mutual translation of the elements.

Preferably the additional free-form surfaces are adapted to provide variable change of additional optical power to provide extension of the depth of field of the lens.

Another attractive embodiment provides the feature that the additional free-form surfaces are adapted to provide variable change of additional optical power to provide reduction of the depth of field of the lens.

It is also possible that the additional free-form surface is adapted to provide astigmatism variable optical power.

It is preferred when the additional free-form surface is adapted to provide aspherical variable optical power.

According to another possibility the additional free-form surface is adapted to provide any variable power of any Zernike mode.

It is also possible that the additional free-form surface is adapted to provide a combination of multiple variable optical powers of any multiple number of Zernike modes.

The invention also provides a Combination of a lens according to the invention with an apparatus adapted for measuring the optical power of an eye to be provided of an artificial lens and adapted for conversion of the measured optical power into an optical power of the artificial lens.

Preferably the apparatus is adapted to perform measurements of the eye to be implanted with the lens including providing measurement of the range of optical power change of at least one variable aberration and to provide conversion of said optical power changes into diopter changes for same aberration provided by at least two free-form optical surfaces of the lens.

It is also possible that the apparatus is adapted to perform measurements of the eye implanted with the lens including providing measurement of the range of diopter change of at least one additional variable aberration, to perform conversion of said diopter changes into diopter changes for same aberrations provided by at least two free-form optical surfaces of the lens, and to perform adaptation of said diopter changes of free-form surfaces by any post-operative free-form change procedure.

According to an embodiment the apparatus is adapted to manufacture the lens before the implantation takes place.

It is however also possible that the apparatus is adapted to perform a post-operative free-form change procedure.

More specifically the apparatus is adapted to perform a post-operative free-form change procedure by laser treatment of the lens in situ.

### BRIEF DESCRIPTION OF THE DRAWINGS

FIG. 1 shows an example of variable extension of depth of field, for an accommodating lens according to the prior art;

FIG. 2 shows an aberrated, fixed optical system according to the prior art; and

FIG. 3 illustrates an accommodating intraocular lens with combination of variable aberrations for extension of depth of field, according to the invention.

### DESCRIPTION OF THE INVENTION

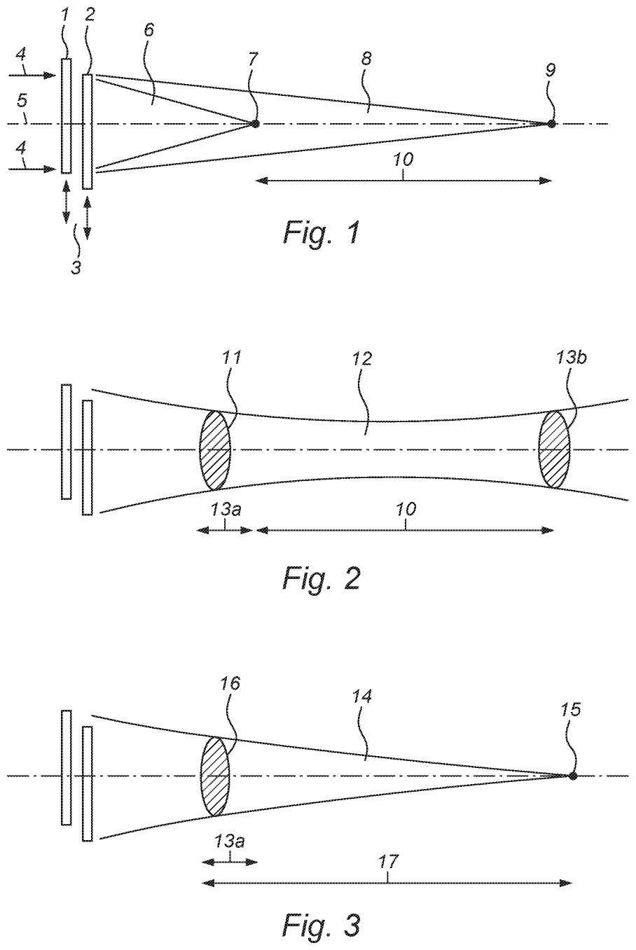

FIG. 1-3 show an example of variable extension of depth of field for an accommodating lens comprising additional free-form surfaces for variable aspherical aberration. FIG. 1-2 illustrate prior art to explain the present invention in FIG. 3.

FIG. 1. A non-aberrated, say, perfect, optical system with sharp focus over the desired range, with an accommodating lens comprising two optical elements, 1, 2, which translate in a direction largely perpendicular to the optical axis, 3, the incoming light beam, 4, the optical axis, 5, the outgoing focused light beam, 6, providing the focal spot for near vision, 7, and, after translation of at least one of the optical element, the outgoing focused light beam, 8, providing the focal spot for far, 9, and the focal range, 10, of the lens.

FIG. 2. An aberrated fixed optical system with a fixed blurred focus, with the blur, 11, adding extended depth of field at near, 13a, which can be desired, but also adding blur to far, 13b, which is generally undesired because vision at far is more sensitive to image degradation compared to vision at near. This figure shows the effect of, for example, a fixed power aspheric addition to a fixed focus lens, over the desired range, with the blur illustrated by a widened focal spot, a focal tunnel, 12.

FIG. 3. This figure illustrates an example of the present invention. A variable aberrated optical system with variable blurred focus, provided by, for example, an variable aspheric addition to a variable focus lens, which results in firstly, blur, 16, extending the desired range at near vision, 13a, and, secondly, by variable correction of blur gradually reducing the focal tunnel into, thirdly, a sharp focal spot, 15 at far vision while maintaining the extended focal range as in FIG. 2, 17.

So, in summary, the present document discloses an accommodating intraocular lens, having an optical axis, with the lens comprising at least two optical elements of which at least one element is adapted to translate in at least one direction largely perpendicular to the optical axis with at least two of the elements each comprising at least one free-form optical surface adapted to provide variable defocus optical power of which the degree of optical power is depending on the degree of mutual shift of the elements with each of the elements also comprising at least one additional free-form optical surface adapted to provide variable optical power of at least one additional aberration other than defocus of which the degree of variable additional aberration optical power is depending on the degree of mutual shift of the elements.

The additional free-form surfaces can provide a variable increase of additional variable optical power depending on the degree of mutual shift of the elements, or, alternatively, the additional free-form surfaces can provide a variable decrease of additional variable optical power depending on the degree of mutual shift of the elements.

For example, the additional free-form surfaces can provide variable change of additional variable optical power to provide extension of the depth of field of the lens, or, alternatively, the additional free-form surfaces can provide variable change of additional variable optical power to provide reduction of the depth of field of the lens.

The additional free-form surface can provide astigmatism variable optical power, or, alternatively, can provide aspherical variable optical power, or, alternatively can provide any variable power of any Zernike mode, or, alternatively, can provide a combination of multiple variable optical powers of any multiple number of Zernike modes.

The method to provide such a lens can be a pre-operative method which includes, firstly, measurements of the eye to be implanted with the lens including providing measurement of the range of diopter change of at least one variable aberration, secondly, provide conversion of said diopter changes into diopter changes for same aberrations provided by at least two free-form optical surfaces of the lens, and, thirdly, provide manufacturing and implantation of the lens. Alternatively, the method to provide such a lens can be a post-operative method which includes, firstly, measurements of the eye implanted with the lens including providing measurement of the range of diopter change of at least one additional variable aberration, secondly, provide conversion of said diopter changes into diopter changes for same aberrations provided by at least two free-form optical surfaces of the lens, and, thirdly, provide adaptation of said diopter changes of free-form surfaces by any post operative free-form change procedure. The post-operative free-form change procedure can be any procedure, for example, post operative modification of intraocular material by laser treatment, for example femtosecond laser treatment.

The method to provide such a lens can provide an increase in diopter change of any aberration per unit of translation of the optical elements, for example, but not restricted to, an increase aspheric aberration to provide a desired extension of depth of field as illustrated in FIGS. 1-3.

Alternatively, the method to provide such a lens can provide a decrease in diopter change of any aberration per unit of translation of the optical elements, for example, a decrease in undesired variable coma or any other undesired variable which degrades visual acuity of the eye.

The invention further relates to a combination of a lens according to the invention and an apparatus adapted for measuring the optical power of an eye to be provided of an artificial lens and adapted for conversion of the measured optical power into an optical power of the artificial lens.

In an embodiment, the apparatus is adapted to perform measurements of the eye to be implanted with the lens including providing measurement of the range of optical power change of at least one variable aberration and to provide conversion of said optical power changes into diopter changes for same aberration provided by at least two free-form optical surfaces of the lens.

In an embodiment (combinable with other embodiments of the invention) the apparatus is adapted to perform measurements of the eye implanted with the lens including providing measurement of the range of diopter change of at least one additional variable aberration, to perform conversion of said diopter changes into diopter changes for same aberrations provided by at least two free-form optical surfaces of the lens, and to perform adaptation of said diopter changes of free-form surfaces by any post operative free-form change procedure.

In an embodiment (combinable with other embodiments of the invention) the apparatus is adapted to manufacture the lens.

In an embodiment (combinable with other embodiments of the invention) apparatus is adapted to perform a post-operative free-form change procedure.

In an embodiment (combinable with other embodiments of the invention) the apparatus is adapted to perform a post-operative free-form change procedure by laser treatment of the lens in situ.

1. An accommodating intraocular lens, having an optical axis, with the lens comprising at least two optical elements, and haptics to allow mutual translation of said elements in a direction substantially perpendicular to the optical axis, in which at least two of the elements each comprising at least one free-form optical surface adapted to provide variable defocus optical power of which the rate of optical power is depending on the degree of mutual translation of the elements wherein each of the elements comprises at least one additional free-form optical surface providing variable optical power of at least one additional aberration other than defocus with the rate of variable additional aberration optical power depending on the degree of mutual translation of the elements.

2. The lens according to claim 1, wherein the additional free-form surfaces are adapted to provide a variable increase of additional optical power depending on the degree or rate of mutual translation of the elements.

3. The lens according to claim 1, wherein the additional free-form surfaces are adapted to provide a variable decrease of additional optical power depending on the degree or rate of mutual translation of the elements.

4. The lens according to claim 2, wherein the additional free-form surfaces are adapted to provide variable change of additional optical power to provide variable extension of the depth of field of the lens.

5. The lens according to claim 2, wherein the additional free-form surfaces are adapted to provide variable change of additional optical power to provide an increase of the depth of field of the lens.

6. The lens according to claim 4, wherein the additional free-form surfaces are adapted to provide variable change of additional optical power to provide a decrease of the depth of field of the lens.

7. The lens according to claim 1, wherein the additional free-form surfaces are adapted to provide

8. The lens according to claim 1, wherein the additional free-form surfaces are adapted to provide any variable power of any Zernike mode.

9. The lens according to claim 1, wherein the additional free-form surfaces are adapted to provide a combination of multiple variable optical powers of any multiple number of Zernike modes.

10-20. (canceled)

21. The lens according to claim 1, wherein the additional free-form surfaces are adapted to provide variable aspherical variable optical power.
